(19) United States
(12) Patent Application Publication
(10) Pub. No.: US 2023/0001145 A1
(43) Pub. Date: Jan. 5, 2023

(54) Non-Integrated Catheter System Having an Extension Set

(71) Applicant: Becton, Dickinson and Company, Franklin Lakes, NJ (US)

(72) Inventors: Jonathan Karl Burkholz, Salt Lake City, UT (US); Weston F. Harding, Lehi, UT (US); Curtis H. Blanchard, Riverton, UT (US)

(21) Appl. No.: 17/852,526

(22) Filed: Jun. 29, 2022

Related U.S. Application Data
(60) Provisional application No. 63/218,161, filed on Jul. 2, 2021.

(51) Int. Cl. A61M 25/00; A61M 25/06; A61B 5/15

(52) U.S. Cl. CPC A61M 25/0097; A61M 25/0606; A61B 5/150992; A61B 5/15003

(57) ABSTRACT

A catheter system may include a catheter adapter, which may include a distal end and a proximal end aligned with the distal end. The catheter system may include a catheter extending from the distal end of the catheter adapter. The catheter system may include an extension set, which may include a distal end, which may include a first connector coupled to the proximal end of the catheter adapter. The extension set may include a second connector and an extension tube disposed between the first connector and the second connector. The catheter, the catheter adapter, the first connector, the extension tube, and the second connector may be in fluid communication. The catheter system may include a stabilization platform disposed below the first connector.

### CROSS-REFERENCE TO RELATED APPLICATION

The present application claims priority to U.S. Provisional Application Ser. No. 63/218,161, entitled “Non-Integrated Catheter System Having an Extension Set”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample. Sometimes an instrument, such as a guidewire or tube, may be inserted through the catheter assembly to reduce or remove occlusions such as the fibrin sheath or thrombus and extend a life of the catheter.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. More particularly, in some embodiments, the present disclosure relates to a catheter system that is non-integrated and includes an extension set. In some embodiments, the present disclosure relates to the extension set and/or related methods.

In some embodiments, a catheter system may include a catheter assembly, which may include a catheter and a catheter adapter. In some embodiments, the catheter adapter may include a distal end and a proximal end aligned with the distal end. In some embodiments, the catheter system may be non-integrated, meaning the catheter adapter of the catheter system may not include a side port with an extension tube integrated with the side port. In these embodiments, the catheter adapter may be referred to as straight. In some embodiments, the catheter extending from the distal end of the catheter adapter. In some embodiments, the catheter may include a peripheral intravenous catheter, a midline catheter, or peripherally inserted central catheter. In some embodiments, the catheter may be inserted through skin of a patient and into vasculature of the patient at an insertion site via an introducer needle, which may be removed after the catheter is positioned within the vasculature.

In some embodiments, the catheter system may include an extension set, which may include a distal end. In some embodiments, the distal end of the extension set may include a first connector coupled to the proximal end of the catheter adapter. In some embodiments, the first connector may include a luer-lock connector, which may be threaded to facilitate securement with the catheter adapter. In some embodiments, the first connector may include a blunt cannula and/or one or more snap features, which may also facilitate securement with the catheter adapter.

In some embodiments, the extension tube may extend from a proximal extension proximate the first connector. In some embodiments, the proximal extension and the first connector may be monolithically formed as a single unit. In some embodiments, the proximal extension may be generally cylindrical. In some embodiments, a port may extend from a side of the proximal extension, and the extension tube may be integrated with the port.

In some embodiments, the catheter system may include a second connector. In some embodiments, the catheter system may include an extension tube disposed between the first connector and the second connector. In some embodiments, a fluid pathway of the catheter system may extend through one or more of following: the catheter, the catheter adapter, the first connector, the extension tube, and the second connector. In some embodiments, the second connector may be configured to couple to an instrument advancement device and may facilitate instrument access to the catheter. In some embodiments, the extension tube may be short, which may decrease a length of an instrument of the instrument advancement device needed to extend through the catheter. In some embodiments, the extension tube and the second connector may provide a remote connection for the instrument advancement device, which may decrease a risk of disturbing the insertion site of the catheter. In some embodiments, the instrument advancement device may be coupled to a blood collection device such that the extension tube and the second connector may also facilitate blood collection at a remote location. In some embodiments, the extension set may include an anti-reflux valve, which may be disposed within the second connector.

In some embodiments, the catheter system may include a stabilization platform, which may be disposed below the first connector for stability and to reduce movement of the catheter. In some embodiments, the stabilization platform may include a first wing and a second wing opposing the first wing, which may facilitate stabilization of the catheter assembly on the skin of the patient and may facilitate taping of the catheter assembly to the skin. In some embodiments, an upper surface of the stabilization platform may be coupled to the luer-lock connector. In some embodiments, the stabilization platform may be overfolded over one or more of the following: the extension tube, the luer-lock connector, and the proximal extension. In these and other embodiments, the stabilization platform may include an elastomeric material. In some embodiments, the stabilization platform may be separately molded and attached to one or more of the following: the extension tube, the luer-lock connector, and the proximal extension.

In some embodiments, a bottom surface of the stabilization platform may be arc-shaped, which may facilitate positioning and securement of the stabilization platform on a curved arm surface of the patient. In some embodiments, the catheter, the catheter adapter, the luer-lock connector, the second connector, and the extension tube may be axially aligned, which may facilitate insertion of the instrument of the instrument advancement device therethrough. In some embodiments, the instrument may include a tube, a guidewire, a probe, or another suitable instrument. In some embodiments, the instrument may include one or more sensors. In some embodiments, the instrument may be configured for blood sampling, fluid administration, sensing, light disinfection, or another purpose.

In some embodiments, the extension set may include another extension tube longer than the extension tube, and the fluid pathway may extend through the other extension tube. In some embodiments, a clamp may be disposed on the other extension tube, and the other extension tube may be clamped off when not in use. Additionally, or alternatively, another clamp may be disposed on the extension tube, and the extension tube may be clamped off when not in use. In some embodiments, the other extension tube may facilitate remote connection with an infusion device or another suitable device, decreasing a risk of disturbing the insertion site of the catheter. In some embodiments, the other extension tube may extend in a proximal direction, which may facilitate movement of fluid from the other extension tube in a distal direction through the catheter assembly.

In some embodiments, a distal end of the other extension tube may be coupled to a third connector, and the third connector may be coupled to the second connector, which may provide a generally straight path for instrument advancement. In some embodiments, the other extension tube may extend from the stabilization platform. In some embodiments, the other extension tube may extend from the second connector. In some embodiments, an angle of the other extension tube with respect to the extension tube may be adjustable. In some embodiments, the angle may be adjustable between 15° and 160°.

In some embodiments, the extension tube may be angled downwardly with respect to a longitudinal axis of the first connector, which may result in a lower profile catheter placement and smooth feeding and advancement of the instrument. In some embodiments, an angle between the longitudinal axis of the first connector and a longitudinal axis of the extension tube may be between 135° and 180°.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example extension set, according to some embodiments;

FIG. 1B is a side view of the extension set of FIG. 1A, according to some embodiments;

FIG. 2A is an upper perspective view of an example extension set, according to some embodiments;

FIG. 2B is a side view of the extension set of FIG. 2A, according to some embodiments;

FIG. 3A is an upper perspective view of an example extension set, according to some embodiments;

FIG. 3B is a side view of the extension set of FIG. 3A, according to some embodiments;

FIG. 4A is an upper perspective view of an example extension set, according to some embodiments;

FIG. 4B is a side view of the extension set of FIG. 4A, according to some embodiments;

FIG. 5A is an upper perspective view of an example extension set, according to some embodiments;

FIG. 5B is a cross-sectional view of the extension set of FIG. 5A, according to some embodiments;

FIG. 5C is a side view of an example extension set, illustrating an example extension tube angled downwardly with respect to a longitudinal axis of an example first connector, according to some embodiments;

FIG. 6A is an upper perspective view of an example extension set, according to some embodiments;

FIG. 6B is an upper perspective view of an example extension set, according to some embodiments;

FIG. 6C is an upper perspective view of an example extension set, according to some embodiments;

FIG. 6D is an upper perspective view of an example extension set, according to some embodiments;

FIG. 7 is an upper perspective view of an example catheter system, according to some embodiments;

FIG. 8 is an upper perspective view of an example catheter system, according to some embodiments;

FIG. 9 is an upper perspective view of an example catheter system, according to some embodiments;

FIG. 10A is an upper perspective view of an example catheter system having a length between a distal end of an example catheter and an example instrument access point, according to some embodiments;

FIG. 10B is an upper perspective view of an example catheter system having the length between a distal end of an example catheter and another example instrument access point, according to some embodiments;

FIG. 11A is an upper perspective view of an example extension set, according to some embodiments; and

FIG. 11B is a side view of the extension set of FIG. 11A, according to some embodiments.

### DETAILED DESCRIPTION

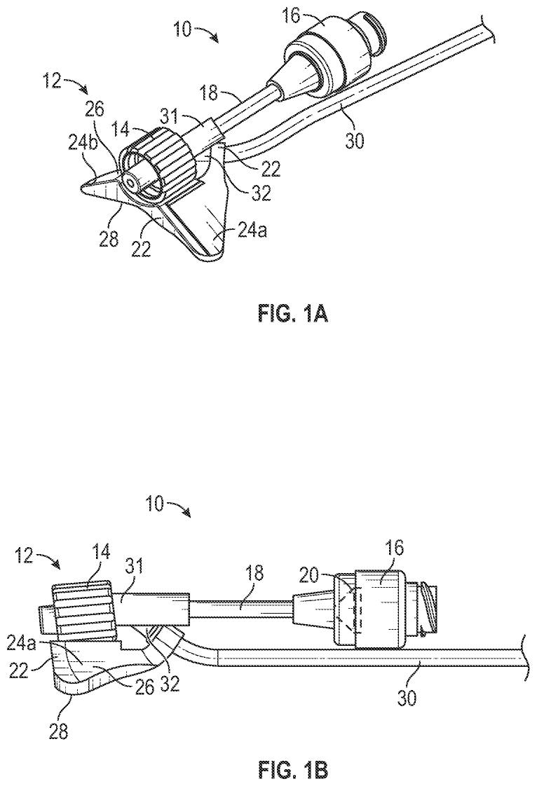

Referring now to FIGS. 1A-1B, in some embodiments, a catheter system may include a catheter assembly, which may include a catheter and a catheter adapter. In some embodiments, the catheter system may include an extension set 10, which may include a distal end 12. In some embodiments, the distal end 12 of the extension set 10 may include a first connector 14, which may be coupled to the proximal end of the catheter adapter. As illustrated in FIGS. 1A-1B, in some embodiments, the first connector 14 may include a luer-lock connector, which may be threaded to facilitate securement with the catheter adapter. In further detail, in some embodiments, the luer-lock connector may include a collar, which may include and inner surface having one or more threads.

In some embodiments, the extension set 10 may include a second connector 16, which may include a female luer connector or another suitable connector. In some embodiments, the second connector 16 may be disposed at a proximal end of the extension set 10. In some embodiments, the catheter system may include an extension tube 18 disposed between the first connector 14 and the second connector 16. In some embodiments, the catheter, the catheter adapter, the first connector 14, the extension tube 18, and the second connector 16 may be in fluid communication. In some embodiments, the second connector 16 may be configured to couple to an instrument advancement device and may facilitate instrument access to the catheter.

In some embodiments, the extension tube 18 may be short, which may decrease a length of an instrument of the instrument advancement device needed to extend through the catheter. In some embodiments, the extension tube 18 and the second connector 16 may provide a remote connection for the instrument advancement device, which may decrease a risk of disturbing an insertion site of the catheter. In some embodiments, the instrument advancement device may be coupled to a blood collection device such that the extension tube 18 and the second connector 16 may also facilitate blood collection at a remote location. In some embodiments, the extension set 10 may include an anti-reflux valve 20, which may be disposed within the second connector 16.

In some embodiments, the extension set 10 may include a stabilization platform 22, which may be disposed below the first connector 14 for stability. In some embodiments, the stabilization platform 22 may include a first wing 24a and a second wing 24b opposing the first wing 24a, which may facilitate stabilization of the catheter assembly on skin of a patient and may facilitate taping of the catheter assembly to the skin. In some embodiments, an upper surface 26 of the stabilization platform 22 may be coupled to the luer-lock connector.

In some embodiments, the stabilization platform 22 may be over molded over one or more of the following: the extension tube 18, the luer-lock connector, and the proximal extension 31. In these and other embodiments, the stabilization platform 22 may include an elastomeric material. In some embodiments, the stabilization platform 22 may be separately molded and attached to one or more of the following: the extension tube 18, the luer-lock connector, and the proximal extension 31. In some embodiments, an entire length of the extension tube 18 between the proximal extension 31 and the second connector 16 may be 1 inch or less, 2 inches or less, 3 inches or less, 4 inches or less, or another suitable length.

In some embodiments, a bottom surface 28 of the stabilization platform 22 may be arc-shaped, which may facilitate positioning and securement of the stabilization platform 22 on a curved arm surface of the patient. In some embodiments, the instrument may include a tube, a guidewire, a probe, or another suitable instrument. In some embodiments, the instrument may include one or more sensors. In some embodiments, the instrument may be configured for blood sampling, fluid administration, sensing, light disinfection, or another purpose.

In some embodiments, the extension set 10 may include another extension tube 30, which may be longer than the extension tube 18. In some embodiments, the fluid pathway may extend through the other extension tube 30. In some embodiments, the other extension tube 30 may facilitate remote connection with an infusion device or another suitable device, decreasing a risk of disturbing the insertion site of the catheter. In some embodiments, the other extension tube 30 may extend in a proximal direction, as illustrated, for example, in FIGS. 1A-1B, which may facilitate movement of fluid from the other extension tube 30 in a distal direction through the catheter assembly.

In some embodiments, the extension tube 18 may extend from a proximal extension 31 proximate the first connector 14. In some embodiments, the proximal extension 31 may be generally cylindrical. In some embodiments, a port 32 may extend from a side of the proximal extension 31, and the extension tube 18 may be integrated with the port 32.

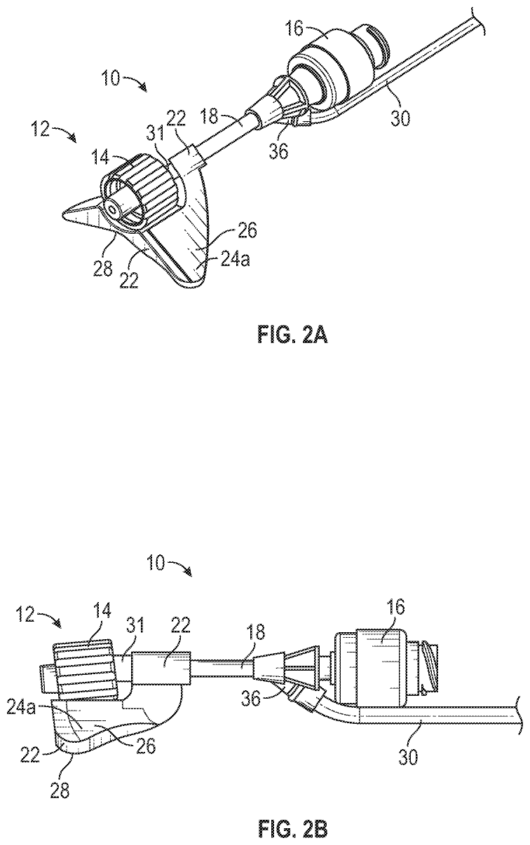

Referring now to FIGS. 2A-2B, the extension set 10 is illustrated, according to some embodiments. In some embodiments, the other extension tube 30 may extend from the second connector 16. FIGS. 2A-2B illustrate the stabilization platform 22 over molded over the extension tube 18 and the proximal extension 31, which may stabilize the extension tube 18, according to some embodiments. In some embodiments, the second connector 16 may include a port 36, and the other extension tube 30 may be integrated within the port 36.

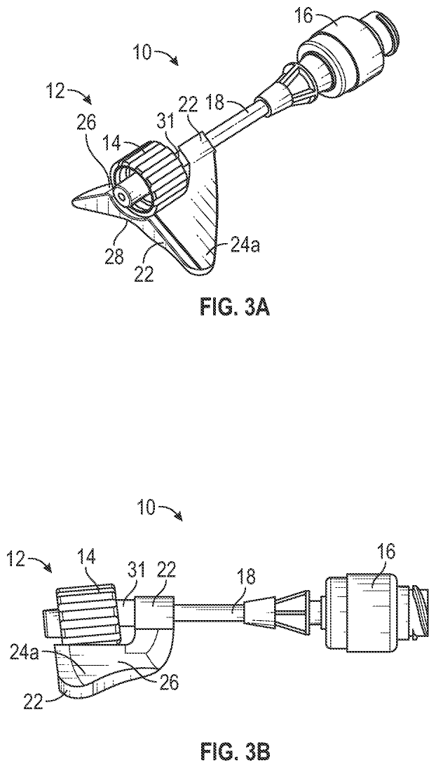

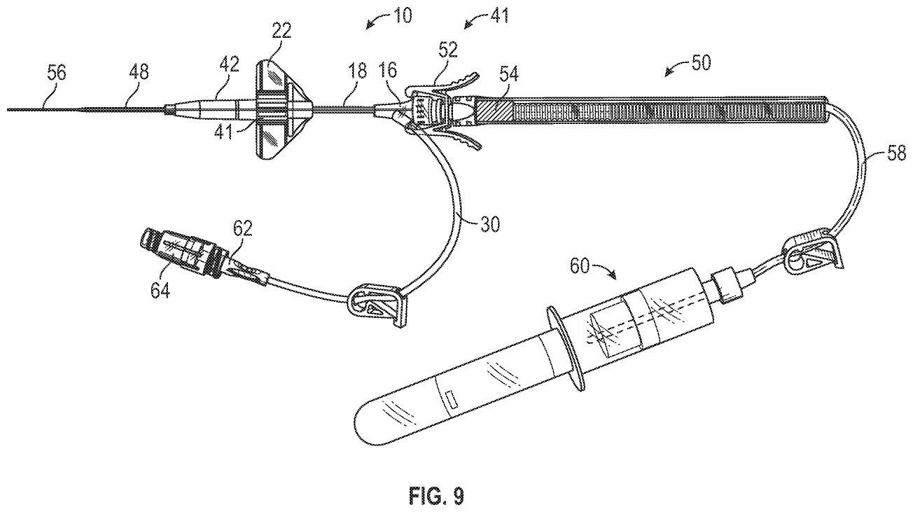

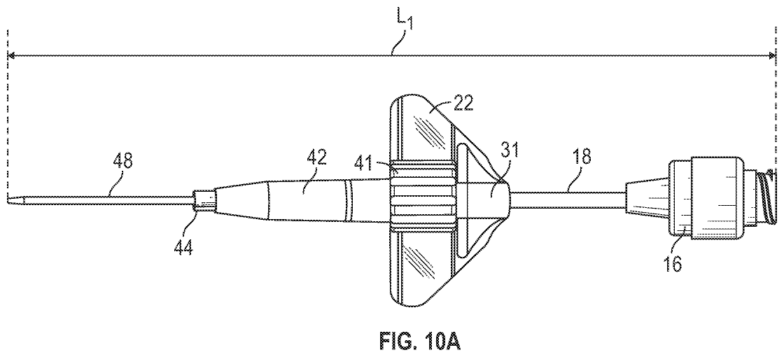

Referring now to FIGS. 3A-3B, in some embodiments, a distal end of the other extension tube 30 may be coupled to a third connector 33, and the third connector 33 may be coupled to the second connector 16. In some embodiments, this configuration may provide a generally straight path for instrument advancement through the other extension tube 30 and the extension tube 18. In some embodiments, a fourth connector (see, for example, FIG. 9) may be disposed at a proximal end of the other extension tube 30. FIGS. 3A-3B illustrate the stabilization platform 22 over molded over the extension tube 18 and the proximal extension 31, which may stabilize the extension tube 18, according to some embodiments.

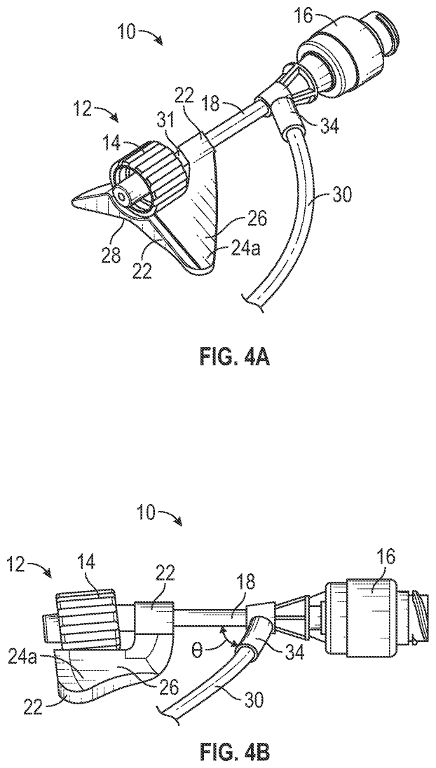

Referring now to FIGS. 4A-4B, in some embodiments, an angle 0 of the other extension tube 30 with respect to the extension tube 18 may be adjustable. In some embodiments, this may be accomplished through a port 34 that includes a ball valve or another suitable mechanism. In some embodiments, the angle θ may be adjustable between about 15° and about 160°. In some embodiments, the other extension tube 30 may be integrated with the port 34.

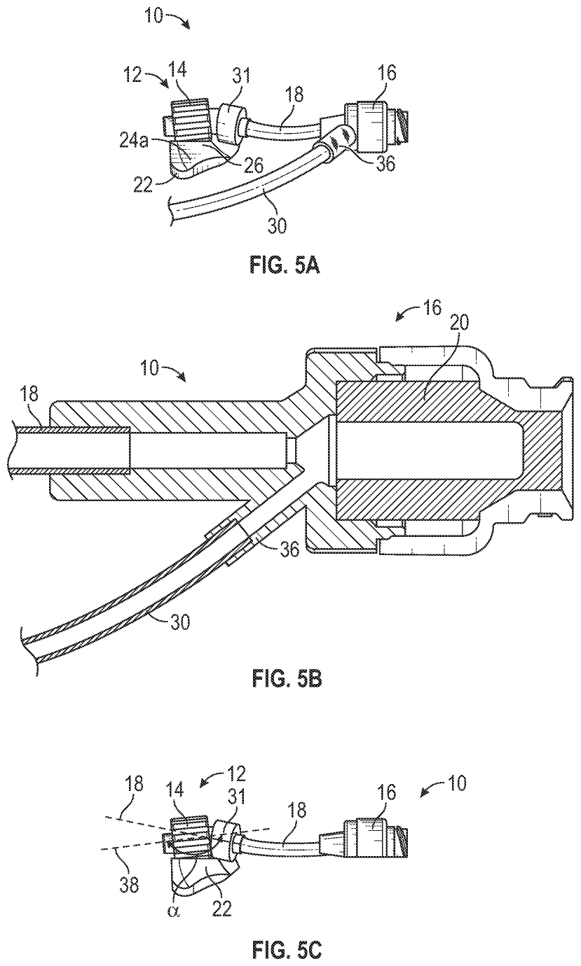

Referring now to FIGS. 5A-5B, in some embodiments, the other extension tube 30 may extend from the second connector 16. In some embodiments, the second connector 16 may include the port 36, which may be fixed and/or integrated with the other extension tube 30. In some embodiments, the port 36 and/or the other extension tube 30 may extend in a distal direction, which may provide clearing of any blood or fluid residue on the anti-reflux valve 20.

Referring now to FIG. 5C, in some embodiments, the extension tube 18 may be angled downwardly with respect to a longitudinal axis 38 of the first connector 14, which may result in a lower profile catheter placement and smooth feeding and advancement of the instrument. In further detail, in some embodiments, an angle α between the longitudinal axis 38 of the first connector 14 and a longitudinal axis 40 of the extension tube 18 may be between 135° and 180°. In some embodiments, the angle α may be greater than 90°. In some embodiments, downward angling of the extension tube 18 with respect to the longitudinal axis 38 may result from downward angling of the proximal extension 31, which may also be disposed at the angle α with respect to the longitudinal axis 38 of the first connector 14. In some embodiments, the extension tube 18 may be flexible, such that the distal end of the extension tube 18 proximate the proximal extension 31 may have the longitudinal axis 40 and may be disposed at the angle α, but a proximal end of the extension tube 18 may not be disposed at the angle α or aligned with the longitudinal axis 40.

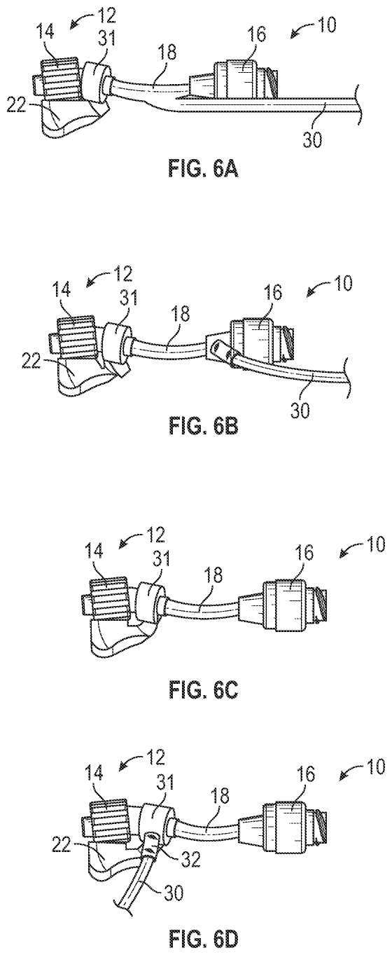

Referring now to FIGS. 6A-6B, several configurations in which the extension tube 18 is angled downwardly with respect to a longitudinal axis 38 of the first connector 14 are illustrated, according to some embodiments. In further detail, in some embodiments, the angle α (see, for example, FIG. 5C) between the longitudinal axis 38 of the first connector 14 and a longitudinal axis 40 of the extension tube 18 may be between 135° and 180° in FIGS. 6A-6D. In some embodiments, the angle α may be greater than 90°.

As illustrated in FIG. 6A, in some embodiments, the other extension tube 30 may extend from the extension tube 18. In these embodiments, the other extension tube 30 and the extension tube 18 may be monolithically formed as a single unit. As illustrated in FIG. 6D, in some embodiments, the other extension tube 30 may extend from the port 32 in a distal direction.

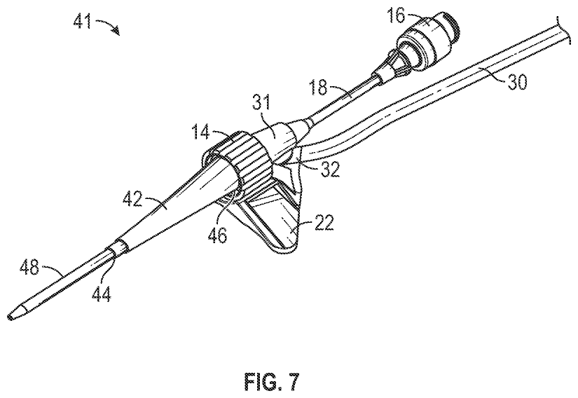

Referring now to FIG. 7, in some embodiments, a catheter system 41 is illustrated, according to some embodiments. In some embodiments, the catheter adapter 42 may include a distal end 44 and a proximal end 46 aligned with the distal end 44. In some embodiments, the catheter system 41 may be non-integrated and straight. In some embodiments, the catheter 48 extending from the distal end 44 of the catheter adapter 42 may be secured within the distal end 44. In some embodiments, the catheter 48 may include a peripheral intravenous catheter, a midline catheter, or peripherally inserted central catheter. In some embodiments, the catheter 48 may be constructed of plastic or another suitable material to reduce potential damage to the vasculature. In some embodiments, the catheter 48 may be inserted through skin of the patient and into vasculature of the patient at an insertion site via an introducer needle (not illustrated), which may be removed after the catheter 48 is positioned within the vasculature.

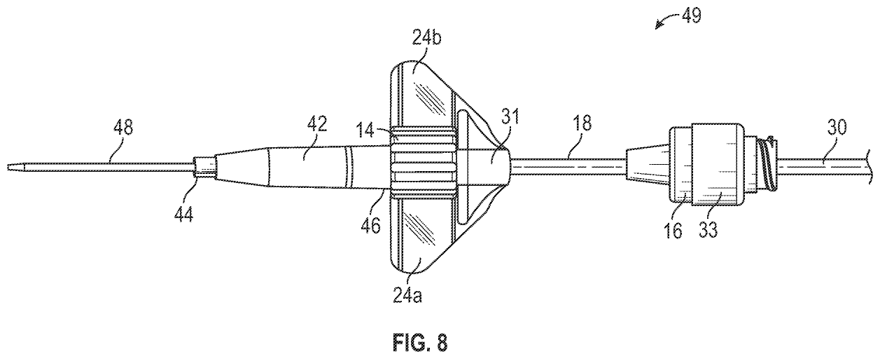

Referring now to FIG. 8, a catheter system 49 is illustrated, according to some embodiments. In some embodiments, the catheter system 49 may be similar or identical to the catheter system 41 in terms of one or more components and/or operation. In some embodiments, proximal edges of the first wing 24a and the second wing 24b may be aligned with each other. In some embodiments, the proximal edges of the first wing 24a and the second wing 24b may be aligned with a distal end of the first connector 14, as illustrated, for example, in FIG. 8.

Referring now to FIG. 9, a catheter system is illustrated, which may be similar or identical to the catheter system 41 and/or the catheter system 49 in terms of one or more components and/or operation. In some embodiments, the catheter system may include an instrument advancement device 50, which may include a distal connector 52 configured to couple to the extension set 10. In some embodiments, the instrument advancement device 50 may include an advancement tab 54 configured to move along a slot in a housing of the instrument advancement device 50 between a proximal or retracted position and a distal or advanced position. In some embodiments, in response to the advancement tab 54 being in the distal position, the instrument 56 may move from inside the housing through the catheter assembly, as illustrated, for example, in FIG. 9.

In some embodiments, the instrument 56 may include a tube that may be coupled to an extension tube 58 that extends through the housing and is coupled to a blood collection device 60. In some embodiments, the blood collection device may include any suitable blood collection device configured to provide suction, such as, for example, a syringe, a BD VACUTAINER® blood collection tube available from Becton Dickinson & Company of Franklin Lakes, N.J. In these and other embodiments, the blood collection device may include a vacuum tube receiver and/or a vacuum tube.

A type of the instrument advancement device 50 may vary. An example of an instrument advancement device is described in further detail in U.S. patent application Ser. No. 17/574,127, filed Jan. 12, 2022, and U.S. patent application Ser. No. 17/701,124, filed Mar. 22, 2022, which are both hereby incorporated by reference in their entirety.

In some embodiments, the fourth connector 62 may be disposed at the proximal end of the other extension tube 30. In some embodiments, a needleless access connector 64 may be coupled to the fourth connector and/or a needleless connector may be disposed at one or more locations within the catheter system. In some embodiments, the second connector 16 may include a needleless connector. In some embodiments, the needleless access connector 64 may be coupled to a proximal end of the second connector 16. In some embodiments, when the needleless access connector 64 is coupled to the proximal end of the second connector 16, the second connector 16 may not include the anti-reflux valve 20 (see, for example, FIGS. 1B and 5B). In these embodiments, the instrument advancement device 50 may be coupled to the needleless access connector 64.

In some embodiments, a clamp may be disposed on the other extension tube 30, and the other extension tube 30 may be clamped off when not in use. Additionally, or alternatively, another clamp may be disposed on the extension tube 18, and the extension tube 18 may be clamped off when not in use. As illustrated in FIG. 9, in some embodiments, one or more of the following may be arranged or configured to be arranged in a straight or generally straight line: the catheter 48, the catheter adapter 42, the luer-lock connector, the second connector 16, and the extension tube 18, which may facilitate insertion of the instrument 56 of the instrument advancement device 50 therethrough. In some embodiments, a pathway through one or more of the catheter 48, the catheter adapter 42, the first connector 14, the extension tube 18, and the second connector 16 may not include any steps or ridges to prevent the instrument 56 from getting caught as the instrument is advanced distally. In some embodiments, a diameter of the pathway may increase at each connection in a distal direction, which may also reduce a risk of the instrument 56 getting caught as the instrument is advanced distally.

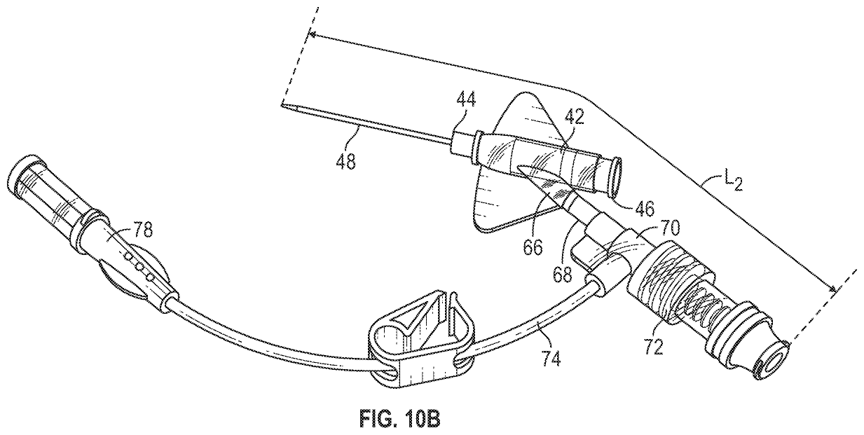

Referring now to FIGS. 10A-10B, a particular catheter assembly may include a length L₁ that extends from a distal tip of the catheter 48 to a proximal end of the second connector 16 or a location where a particular instrument advancement device may be coupled. In some embodiments, the length L₁ may be equal to a length L₂ of an integrated catheter assembly, illustrated, for example, in FIG. 10B. Thus, the length L₁ may be standardized such that the particular instrument advancement device may be configured to advance the instrument to a same location, such as beyond the distal tip of the catheter 48, in both a non-integrated catheter assembly and an integrated catheter assembly.

In some embodiments, the catheter adapter 42 of the integrated catheter assembly may include a side port 66 and an extension tube 68 extending from the side port 66. In some embodiments, a T-connector 70 or a Y-connector may be coupled to a proximal end of the extension tube 68. In some embodiments, a needleless access connector 72 may be coupled to the T-connector 70 and configured to couple to the particular instrument advancement device. In some embodiments, another extension tube 74 may extend from the T-connector 70 and may be coupled to a proximal connector 78. In some embodiments, the extension tube 18 may be a different length based on a length of a particular catheter with which the extension tube 18 may be used.

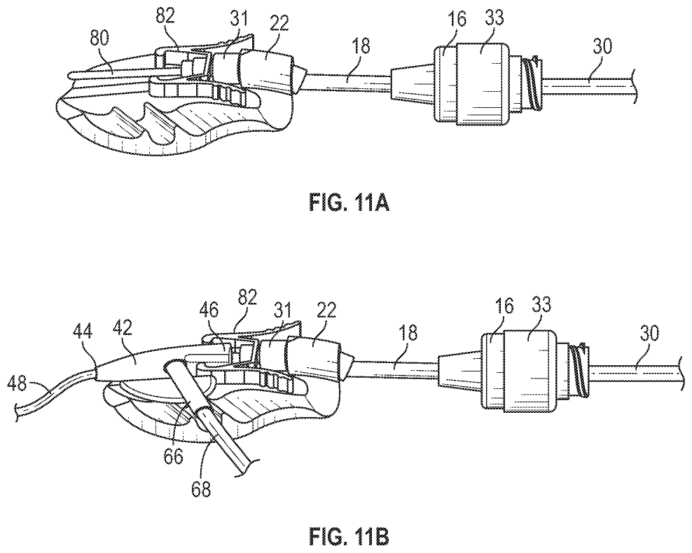

Referring now to FIGS. 11A-11B, in some embodiments, the first connector 14 may include a blunt cannula 80 and/or one or more snap features 82, which may also facilitate securement with the catheter adapter 42. In some embodiments, the blunt cannula 80 may be constructed of metal. In some embodiments, a shape or configuration of the snap features 82 may vary. In some embodiments, the platform 22 may include one or more depressions configured to receive one or more of the following: the first wing 24a, the second wing 24b, the extension tube 68, the side port 66, and the catheter adapter 42, which may each be nested within the depressions.

It is understood that embodiments of FIGS. 1-11 may be combined. For example, the embodiment of FIGS. 1A-1B may be similar or identical to the embodiment of FIGS. 2A-2B in terms of one or more features and/or operation. All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A catheter system, comprising:
a catheter adapter, comprising a distal end and a proximal end aligned with the distal end;
a catheter extending from the distal end of the catheter adapter;
an extension set, comprising:
a distal end, comprising a first connector coupled to the proximal end of the catheter adapter;
a second connector;
an extension tube disposed between the first connector and the second connector, wherein the catheter, the catheter adapter, the first connector, the extension tube, and the second connector are in fluid communication; and
a stabilization platform disposed below the first connector.

2. The catheter system of claim 1, wherein the first connector comprises a luer-lock connector.

3. The catheter system of claim 2, wherein the stabilization platform comprises a first wing and a second wing opposing the first wing.

4. The catheter system of claim 2, wherein an upper surface of the stabilization platform is coupled to the luer-lock connector.

5. The catheter system of claim 2, wherein a bottom surface of the stabilization platform is arc-shaped.

6. The catheter system of claim 2, wherein the catheter, the catheter adapter, the luer-lock connector, the second connector, and the extension tube are configured to be arranged in a straight line.

7. The catheter system of claim 2, wherein the extension set further comprises another extension tube longer than the extension tube, wherein the other extension tube is in fluid communication with the catheter, the catheter adapter, the first connector, the extension tube, and the second extension tube.

8. The catheter system of claim 7, wherein a distal end of the other extension tube is coupled to a third connector, wherein the third connector is coupled to the second connector.

9. The catheter system of claim 7, wherein the extension set further comprises a proximal extension proximate the luer-connector, wherein the extension set further comprises a port extending from a side of the proximal extension, wherein the other extension tube extends from the port.

10. The catheter system of claim 7, wherein the other extension tube extends from the second connector.

11. The catheter system of claim 10, wherein an angle of the other extension tube with respect to the extension tube is adjustable.

12. The catheter system of claim 11, wherein the angle is between 15° and 160°.

13. The catheter system of claim 7, wherein the other extension tube extends from the extension tube.

14. The catheter system of claim 1, wherein the stabilization platform is over molded over the extension tube.

15. The catheter system of claim 1, wherein the extension tube is angled downwardly with respect to a longitudinal axis of the first connector.

16. The catheter system of claim 15, wherein an angle between the longitudinal axis of the first connector and a longitudinal axis of the extension tube is between 135° and 180°.

17. The catheter system of claim 1, wherein the extension set further comprises an anti-reflux valve disposed within the second connector.

18. The catheter system of claim 1, wherein the second connector is configured to couple to an instrument advancement device.

19. The catheter system of claim 1, wherein the first connector comprises a blunt cannula.

20. The catheter system of claim 19, wherein the first connector further comprises a plurality of snap features.
